(19) United States
(12) Patent Application Publication
(10) Pub. No.: US 2015/0306246 A1
(43) Pub. Date: Oct. 29, 2015

(54) MAGNETIC NANOPARTICLES, COMPOSITES, SUSPENSIONS AND COLLOIDS WITH HIGH SPECIFIC ABSORPTION RATE (SAR)

(71) Applicant: THE TRUSTEES OF DARTMOUTH COLLEGE, Hanover, NH (US)

(72) Inventors: Ian Baker, Etna, NH (US); Katsiaryna Kekalo, Lebanon, NH (US)

(73) Assignee: THE TRUSTEES OF DARTMOUTH COLLEGE, Hanover, NH (US)

(21) Appl. No.: 14/650,239

(22) PCT Filed: Dec. 6, 2013
(86) PCT No.: PCT/US2013/073629

§ 371 Date: Jun. 5, 2015

Related U.S. Application Data
(60) Provisional application No. 61/734,831, filed on Dec. 7, 2012.
(60) Provisional application No. 61/911,260, filed on Dec. 3, 2013.

(51) Int. Cl. A61K 47/48

(52) U.S. Cl. CPC A61K 47/48861; B82Y 40/00

(57) ABSTRACT

Iron oxide nanoparticles and nanocomposites with organic molecules embedded in their structure, having exceptionally high SAR values, are provided for biological, medical (for example, drug delivery, hyperthermia, etc.) and other uses.

### CROSS-REFERENCE TO RELATED APPLICATIONS

This application claims the benefit of priority from Provisional Application Ser. Nos. 61/734,831 filed Dec. 7, 2012 and 61/911,260 filed Dec. 3, 2013. Both of the aforementioned applications are incorporated herein by reference in their entireties.

### U.S. GOVERNMENT RIGHTS

This invention was made with government support under Contact No. 5U54CA15662-03 awarded by the National Institute of Health. The government has certain rights in the invention.

### BACKGROUND

Magnetic hyperthermia, which is sometimes also called thermotherapy, operates on the principle that magnetic nanoparticles produce heat when subjected to an alternating magnetic field of suitable frequency and amplitude. This effect may cause, for example, the temperature inside a tumor to rise to therapeutic levels if the nanoparticles are injected into a tumor. Magnetic nanoparticles injected directly into a tumor and heated with an alternating magnetic field have been shown to destroy cancer cells. Such magnetic hyperthermia treatments can also enhance the effects of subsequent radiation therapy or chemotherapy. With nanoparticles localized at the tumor, magnetic hyperthermia can provide treatment of the tumor while leaving surrounding healthy tissue with minimal damage. A key issue with magnetic nanoparticles is that they, having certain composition, size and shape, have a high specific absorption rate (SAR) so that not only is the dose of nanoparticles required for hyperthermia treatment minimized, but also so that lower values of the product of magnetic field strength and frequency are used.

The heating effect of these magnetic nanoparticles is associated with number of phenomena, including the well-known magnetic hysteresis phenomenon. This is demonstrated in the form of a hysteresis loop that results from placing the nanoparticles in a magnetic field which changes direction over time. The area of the loop represents thermal energy that, in consequence of cycling magnetic field, may be absorbed from the field and dissipated into the environment. This energy may be defined as power that is expressed as the specific absorption rate (“SAR”):

SAR=Af,

Where f is the frequency of the magnetic field, and A is the specific loss of the material under study and corresponds to the area of the hysteresis loop.

In selecting nanoparticles for cancer treatment, ones with the highest SAR are much preferred. Having a large SAR value not only minimizes the dose of nanoparticles required for hyperthermia treatment, but is also a key parameter in the decreasing of size of tumor which can be treated. There is also a limit to the concentration of nanoparticles that a cell can take up.

The Stoner-Wohlfarth model is sometimes uses to approximate the effects of magnetization reversal in single domain particles. Stoner, E. C.; Wohlfarth, E. P. (1948). “A mechanism of magnetic hysteresis in heterogeneous alloys” [1]. The size of nanoparticles influences the number of magnetic domains. Where the larger particles have multiple domains, the model is frequently that of a Rayleigh loop.

The most widely used nanoparticles for hyperthermia applications are iron oxide particles. These are presumptively biocompatible and stable against further oxidation. Iron and cobalt particles may advantageously have higher SAR values, but problems exist with respect to toxicity and instability. The relatively lower SAR values of available iron oxide nanoparticles require the use of large quantity them. This is problematic in the sense that cells have a limited uptake capacity, the use of magnetic fields with higher amplitude is generally undesirable or practically unattainable, and these limits constrain the perceived therapeutic applications.

Widely known and used methods of synthesis magnetic nanoparticles are based on: a) mechanical dispersion [2]; b) precipitation of iron oxides [3], c) thermal decomposition [4], d) microemulsion [5] and e) flame spray synthesis [6]. The obtained nanoparticles are decorated further with stabilizers or other type of functional molecules.

### SUMMARY

The presently disclosed instrumentalities advance the art by providing magnetic nanoparticles with significantly improved SAR values. In one embodiment, one embodiment, the particle synthesis includes precipitation of iron oxides and hydroxides in the presence of carbohydrates or other organic chain materials followed by hydrothermal treatment. The ratio of Fe(II):Fe(III) may vary, but is greater than 1:2. It has been discovered, according to one aspect of what is described herein, that suitable ratios of Fe(II) to Fe(III) result in oxidation to form iron oxides and hydroxides. These materials at nanoscale tend to form agglomerants that are collodially stable yet, by way of example, are responsive under the action of a magnetic field to produce a representative SAR up to 600.0 W/g in a frequency range from 100 Hz-200 kHz at applied field strengths ranging from 10-1500 Oe.

In another aspect of what is disclosed, the particles have small sizes that may penetrate cell membranes and tissues. With nanoparticles localized at the tumor, magnetic hyperthermia provides treatment of the tumor while leaving surrounding healthy tissue with minimal damage. Specific materials among those disclosed produce significantly more heat than commercially-available MNPs at 300-400 Oe. Even more valuable is the fact that they produce enough heat for therapeutic treatment at magnetic field strengths as low as 100-200 Oe while commercially-available MNPs do not. Composites and dispersions using these particles may be used for direct and/or systemic injections. The high SAR values improve the ability of these composites to heat at very low field strength and so constitute a revolution in modern world of hyperthermia.

According to a method embodiment, a method of synthesizing MNP includes forming a solution of iron salts wherein the iron salts include a mixture of Fe(II) and Fe(III) in a ratio of Fe(II):Fe(III) greater than 1:2. The iron salts in alkali solution form iron oxides and hydroxides. This is followed by hydrothermally developing crystals in the solution, where the crystals include the iron oxides and hydroxides that may be precipitated from solution.

In one aspect, the crystals present a crystal matrix structure. This may form a nanocomposite where the solution of iron salts further contains an organic chain material, such that the crystals grown in the step of precipitating contain this organic chain material interwoven or interacting in other way with the crystal matrix.

In one aspect, dopants (Me) are optionally added, such as Eu, Co, Zn, Mn, Pt and the like to form such composite ferrites as MeₓFe₁₋ₓO₄, where Do is a dopant metal and x is a number from 0 to 1.

In one aspect, the MNP material may have an average single crystal diameter of from 2-5 nm, and these crystals when suspended as colloids may form aggregates with an average diameter of from 10-100 nm (TEM).

In one aspect, the nanocomposite materials may be decorated with a bioactive agent, such as antibodies, drugs, toxins, markers, others, and combinations thereof. This may be done on commercial order according to processes known to the art.

In one aspect, synthesis may be controlled to produce a Z-size of the composite particles that is dominantly from 70-150 nm.

In one aspect of the disclosure, iron oxide nanoparticles are used as therapeutic tools for the treatment of cancerous tissue, either directly by localized magnetic hyperthermia or when used as a thermal trigger for therapeutic drugs delivered via vesicles.

In one embodiment, the nanoparticles may be bonded with organic molecules (for example, carbohydrates) for improved utility in biological and other applications. Organic molecules implanted or embedded in particle structure prevents particles from losing coating, and thus avoids one of main problems of commercially available magnetic nanoparticles. Chemical modification the magnetic nanoparticles is thereby avoided with also an increase in shelf life.

In one aspect, a method of synthesis is provided that advantageously does not require extra high pressure, which in the prior art may be up to 1000 bar [7].

### BRIEF DESCRIPTION OF THE DRAWINGS

FIG. 1 shows the manner of MNP@Organic molecule formation with organic molecule (for example, carbohydrate) chains embedded in the crystalline structure of MNP.

FIG. 2 includes TEM pictures taken from a bottom fraction of MNP@CM-Dex-40 (FIG. 2(a)) together with the aggregate size distribution for this material (FIG. 2(c)); as well as TEM pictures taken from a top fraction of MNP@CM-Dex-40 (FIG. 2(b)) together with the aggregate size distribution for this material (FIG. 2(d)).

FIG. 3 shows the Z-size of MNP@CM-dex-4 from a bottom fraction (FIG. 3(a) and an upper fraction (FIG. 3(b)).

FIG. 4 shows various magnetization curves of MNP@CM-dex-40.

FIG. 5 shows actual heating behavior of commercial available MNP (Micromode, BNF-starch) (FIG. 5(a)) and MNP produced according to this disclosure. In this particular case the figure shows the upper fraction of MNP@CM-Dex-40 (FIG. 5(b)) and the bottom fraction of MNP@CM-Dex-40 (FIG. 5(c)).

FIG. 6 shows a comparison of SAR values obtained from magnetic nanoparticles obtained by use of the instrumentalities disclosed herein versus magnetic nanoparticles according to the closest approximation of the prior art, i.e. a graphical comparison of SAR performance between commercially available MNP (Micromode, BNF-starch) (FIG. 6(a)) and Dartmouth-invented MNP. In this particular case top fraction of MNP@CM-Dex-40 (FIG. 6(b)) bottom fraction of MNP@CM-Dex-40 (FIG. 6(c)).

FIG. 7 is a process diagram that shows synthesis of MNP according to one embodiment.

### DETAILED DESCRIPTION

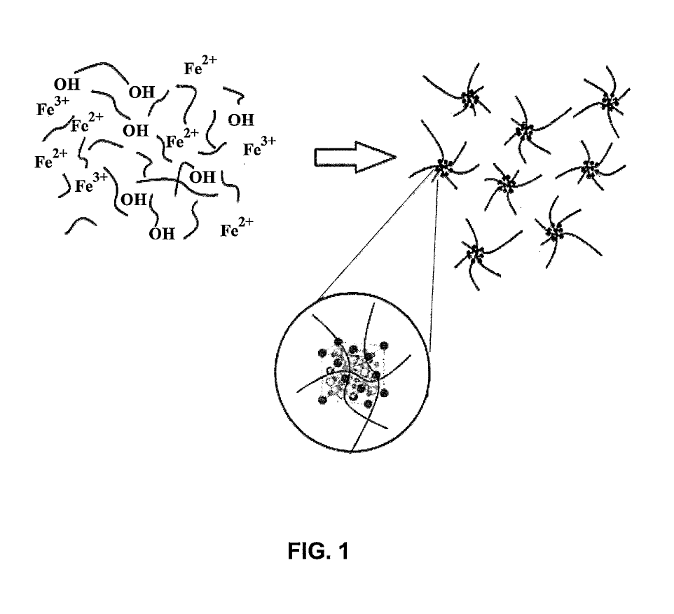

FIG. 1 shows formation of magnetic nanoparticles (MNP) with organic chain molecules (for example, carbohydrate) chains embedded in a crystalline structure formed of iron oxide and iron hydroxide materials. The crystalline structure is b.c.c. in the case of a ferrite (inverse spinel).

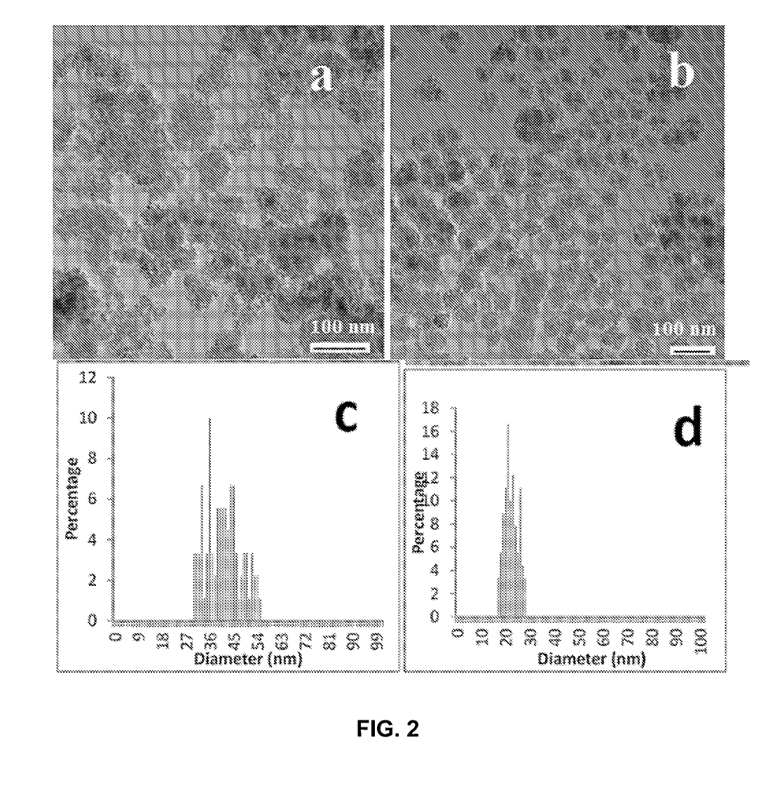

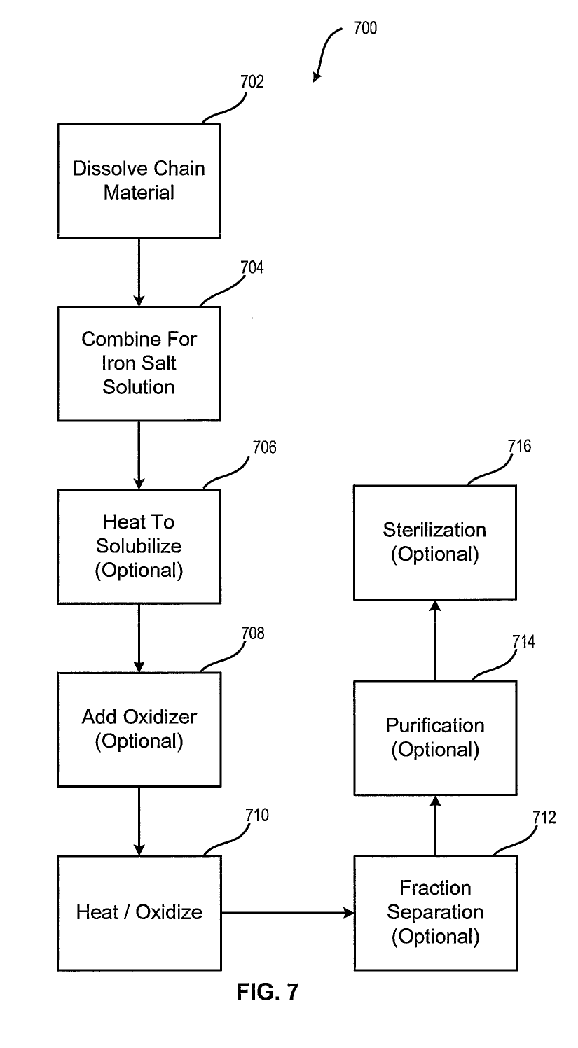

MNP with high SAR may be synthesized according to the instrumentalities disclosed herein. FIG. 7 shows a process 700 of making MNP according to one embodiment. It will be appreciated that FIG. 2 together with this discussion thereof teaches by way of example, and not by limitation.

Step 702 entails forming a solution by dissolving an organic chain material in water or another polar solvent. The organic chain material may be selected from different classes of materials. The organic chain material may include, but not limited to, saccharide, such as a monosaccharaide including for example glucose, mannose, etc.; such disaccharides as sucrose, maltose etc., such polysaccharides as dextran, starch etc.; saccharide derivatives including especially amino-, aminodextrane, etc., carboxy-, caboxymethyl- etc., and other saccharide materials. The organic chain material may also be an alcohol, diol or polyol having a carbon number of two or higher, such as polyethylene glycol. The organic chain material may be an organosilicate, such as tetraethyl orthosilicate, or an organosiloxane, such as 3-aminopropyl)trimethoxysilane, or derivatized versions of these materials. By way of example, organic chain materials may include: dextrans at 6 k, 9.3 k, 40 k, 70 k; glucose; sucrose and starch, dextran derivatives such as carboxymethyl-dextran (CM-DEX) 4 k, 40 k, 70 k; either individually or in any combination.

The amount of organic chain material may vary as a weight percentage of the total mixture, but it is preferred to use an amount that is close to the solubility limit of the chain material in the solvent at temperature. For example, this may be an amount that is 5%, 10%, 15%, 20%, 25%, or 30% less than the amount of the same organic chain material at the that is solubility limit, determined as a percent difference based upon the weight of material at the solubility limit. These organic chain materials may also be used in any combination, in which case this percentage difference is determined on the basis of the organic chain material with the lowest solubility. This percentage is preferably, but not necessarily, determined using a temperature of less than about 50%. The temperature is more preferably less than about 30° C. In one example, this is performed in deionized water, or water solutions containing other chemicals. Ambient or room temperature or is most preferred, and the temperature may be even colder, even down to 0° C. for deionized water.

In Step 704, an iron salts solution containing iron salt of Fe(II) or combination of Fe(II) and Fe(III) salts with a Fe(II) : Fe(III) molar ratio greater than 1:2 is combined with the organic chain material solution of Step 702, preferably with vigorous stirring or mixing. The ratio of greater than 1:2 is intended to produce a combination of magnetite and ferrite, whereas the ratio of 1:2 or lower will result in dominantly ferrite. A ratio of at least 2:1 is preferred, 3:1 is more preferred, and 5:1 is even more preferred for many applications, and even higher ratios may be used.

The iron salts precipitate to form iron oxides and hydroxides, which are referred to herein below as MNP. The iron salts are provided in a sufficient amount to provide, upon substantial completion of the oxidation reaction, an amount of MNP as a weight ratio of MNP to chain material that suitably varies from 1:0.1 to 1:20, although higher or lower weight ratios may also be used. If needed, salts of one or more dopant metals (Me) especially Eu, Co, Zn, Mn, Pt, etc., (and combinations thereof) may optionally also be mixed with the iron solution in amounts of 0-100% determined as atomic percent based upon total amount of iron. The atomic percent amount is preferably an amount of from 0.1% to 3% determined as Me/(Me+Fe(II)) in a structure MeₓFe₁₋ₓFe₂O₃. An amount of 1% by weight dopant is preferred for many applications.

The mixing order of materials is not critical as to the order of mixing, such that the solution described in Step 702 may be added to a pre-mixed iron salt solution of the type described in Step 704, or vice-versa. It is also possible to add the iron salts directly to the solution of Step 702 without premixing, or the organic chain material may be added direct to the iron salt solution, etc.

Step 706 is an optional step that does not need to be performed unless not all of the materials combined in Steps 702 and 704 have dissolved. Heating may occur to any temperature as needed to solubilize the materials.

Step 708 optionally proceeds with the addition of an oxidizer to commence an oxidation reaction that completely or partial converts the iron (II) in solution into iron (III) oxides and hydroxides. While some form of oxidation is essential, this Step may proceed in an optional sense without the addition of chemicals by the simple expedient of exposure to ambient oxygen in the solution or ambient air. Oxidizing gas may be added, such as by the bubbling of oxygen, ozone, or nitrous oxide through the solution. The reaction proceeds more controllably, but also to completion, by the addition of a chemical oxidizing agent, such as a nitrate, nitrite, peroxide, perchlorate, permanganate, persulfate, hypochlorite, sodium nitrate, sodium nitrate, ammonium nitrate, organic oxidizer such as trimethylamine N-oxide or another oxidizer.

A sufficient amount of oxidizing agent is added to drive the oxidation reaction to substantial completion. In the case of a nitrate as represented by sodium nitrate, a 5:1 molar ratio of Fe(II):NaNO₃ is preferred. At this time, alkaline material is optionally added to raise the pH. This may be suitably a hydroxide, such as ammonium hydroxide, sodium hydroxide, potassium hydroxide or other chemical. Maintenance of a basic pH helps iron oxides and hydroxides to form good crystalline structure. A pH of 10 or greater is preferred.

Step 710 includes heating to facilitate the oxidation reaction with resultant particle formation. Crystals as shown in FIG. 1 may be raised, for example, at a temperature of from 20 to 100° C. or higher. This time may range, for example, from five minutes to three hours and longer. Precipitation temperature may be suitably from 0 to 100° C. The rate of heating affects mostly the particle size distribution and crystallinity. Generally speaking, the temperature is ramped up to a target maximum over a predetermined period of time. Crystal growth may be done instantly or prolonged up to 3 hours and longer at this temperature range. This may be suitably, for example, a ramp of from 1° C. to 30° C. per hour, or another ramp rate. This is followed by a period of slow cooling down to a target temperature for cooling. The precipitation is usually performed under close to normal atmospheric pressure. However other pressures (negative or positive) could be applied as well, especially to increase the maximum target temperature range. The media for precipitation contains organic molecules that are to be implanted in magnetic nanoparticle structure. By way of example, excellent results are usually obtained using a target maximum temperature of 100° C., which is ramped from room temperature at a rate of speed 10° C/hour. The hot solution is then left to stand without heat for cooling to the room temperature.

In Step 712, fraction separation is optionally done, for example, via magnetic field application and/or centrifugation to separate bands of particle sizes into different fractions, while removing also large aggregates.

Step 714 includes purifying the particles by eliminating impurities and excess of reactants. Purification does not need to be done in all instance, and may be omitted depending upon the intended use of the particles. Purification may be performed on a Spectrumlab™ dialysis system, for example, by washing particles with 1 L of PBS buffer (1×), then 1 L of DI water. Purification of the particles may be performed by techniques including, for example, magnetic decantation, filtration, centrifugation, dialysis, magnetic columns and others.

Sterilization (Step 716) may be performed if needed by washing with alkali and sterile and endotoxin free water and saline solutions. Other sterilization techniques known to the art could be used as well.

The synthesis is repeatably controllable to provide nanoparticles with a crystal size ranging from 2-5 nm with 10-100 nm aggregates. The Z-size ranges are typically from 70-150 nm.

The formed nanocomposites may be further modified with a wide range of functional molecules including, without limitation, antibodies, drugs, etc. The obtained materials may be provided in form of powder, suspension or colloid solution. Magnetic nanopowders may be resuspended in water to obtain desired concentration. The concentration of colloid solutions may be up to 50% w/w and higher. Colloid solutions have a shelf life of over one year.

The composites have high SAR values (up to 600 W/g) in a wide frequency and field strength range. For example, suitable frequencies include, but not limited to, the range from 100 Hz-200 kHz and other. Suitable field strengths include, without limitation, those from 10-1500 Oe and other. The precipitated nanocomposites may be redispersed in a liquid, such as water, saline solution, plasma, serum and other compatible liquids.

### EXAMPLE 1

Magnetic Nanoparticles Synthesis

Magnetic nanoparticles with organic molecules (in this example mono-polysaccharides or their derivatives) embedded in their structure may be obtained as described above.

To make the synthesized MNP, commercially available ferric chloride (FeCl₃.6H₂O), ferrous sulfate (FeSO₄.7H₂O), 25 wt % ammonium hydroxide solution, NaNO₃, NaOH, starches, glucose and sucrose were purchased from VWR. CM-dextrans (CM-Dex) of different molecular mass were purchased from TdB Consultancy AB. Hydroxyethyl starches (HES) of different molecular weight were purchased from Serumwerk Bernburg AG. All reactants were used as received without further purification. The following synthesis is reported in reference to process steps from FIG. 7, as discussed also above.

Step 702. Forming a saccharide solution by dissolving mono-, polysaccharides or their derivatives in deionized (DI) water to make a 15 w % saccharide solution. In this example we used carboxymethyl dextran 40 k.

In Step 704, an iron solution containing 10 w % iron salts with a Fe(II):Fe(III) molar ratio of 5:1 is added quickly under vigorous stirring into the saccharide solution. In this example the dopant of 1% Eu of Eu/(Eu+Fe) was added.

Step 706 entails heating the resultant mixture to about 70° C.

In Step 708 sodium nitrate was added to the heated solution at a molar ratio of Fe(II): NaNO₃ of 5:1. Sodium hydroxide is also added to maintain pH higher than 10.

Step 710 includes heating to ramp the temperature up to 100° C. at a rate of speed 10° C/hour, then letting stand without heat for cooling to the room temperature.

In Step 712, fraction separation was done via magnetic field application. The bottom fraction is marked as “a”, upper fraction is marked as “c”. This was followed by centrifugation for 15 min at 5000 rpm to remove large aggregates.

Step 714. Purification was performed on a Spectrumlab™ dialysis system, by washing particles with 1 L of PBS buffer (1×), then 1 L of DI water. Sterilization (Step 716) was performed by washing with alkali and sterile and endotoxin free water and saline solutions. This process results in the production of nanoparticles as shown in FIG. 1 and FIG. 2. The obtained materials may be provided in form of powder, suspension or colloid solution. Magnetic nanopowders may be resuspended in water to obtain desired concentration. The concentration of colloid solutions may be up to 50% w/w and higher. Colloid solutions have shelf life of over 1 year.

### EXAMPLE 2

Characterization of Synthesized Nanoparticles

Size Characterization

Transmission electron micrographs (TEM) were taken of nanoparticles that were synthesized according to process 200. This was done using a FEI Technai F20ST field emission gun transmission electron microscope (TEM) operated at 200 kV. The quasi-static magnetic properties of the nanoparticles were determined including magnetic saturation (Ms), remanence magnetization (Mr), and coercivity (Hc) from hysteresis loop measurements using a Lakeshore model 7300 vibrating sample magnetometer (VSM). Specific absorption rate (SAR) was calculated based on exothermal effect recorded during treating of synthesized samples with alternative magnetic field.

Field amplitudes of 50, 100, 200, 300 and 400 Oe at a frequency of 134.5 KHz were applied using a home-made device with generator, amplifier and cooling system for cooling the coil to 20 ° C. The water-cooled copper solenoid coil had a 32 mm inner diameter and was 80 mm in length. Fiber optic temperature probe was positioned to measure temperature in the sample. The sample was placed at the middle of the coil, where the field strength was greatest. Temperature was recorded at one-second intervals throughout the experimental period and monitored in real-time via software supplied by the temperature monitoring system.

The TEM results show that the synthesis is repeatably controllable to provide nanoparticles with a crystal size ranging from 2-5 nm regardless of the nature of saccharide. For the bottom fraction the average size of aggregates (FIG. 2(a), 2(c)) is 40 nm, for the upper fraction (FIG. 2(b), 2(d)) the average size is 20 nm.

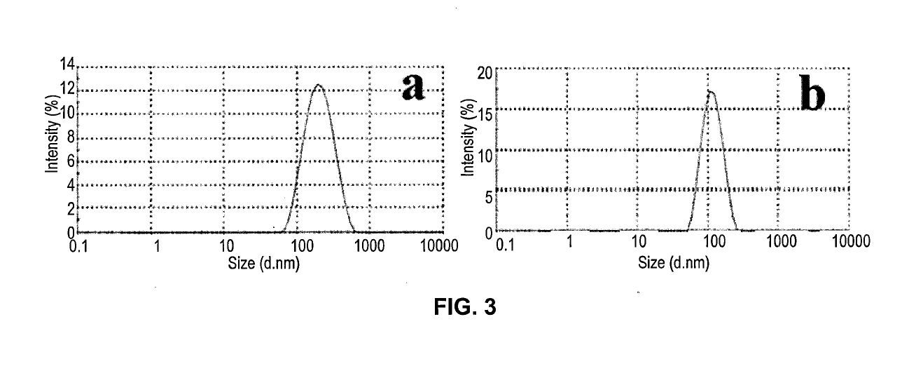

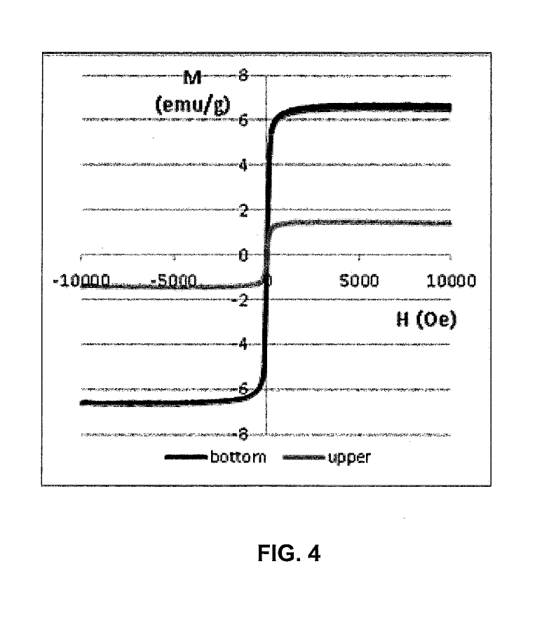

The z-size ranges from 10-800 nm. A typical Z-size distribution is shown in FIG. 3 for MNP@CM-Dex-40 where FIG. 4a shows this for the bottom fraction and FIG. 4b the upper fraction.

Magnetic Properties

The magnetization curves of FIG. 4 show superparamagnetic behavior for the samples. Low saturation magnetization is caused by small MNP size and correlates very well with literature data [8].

Heating Properties

MNP prepared with monosaccharide material lack any heating properties. At the same time MNP with di- and poly-saccharides show very good heating properties and have SAR as high as 344.0 W/g in some cases. Thus, In order to be able to perform hyperthermia by systematic as well as direct injection and to improve also cellular uptake of MNP, it is preferred to utilize MNP with polysaccharide derivatives such as CM-dex and HES. In these cases, MNP have functional groups and antibodies may be easily attached to them.

SAR values of MNP@HES show that decreasing the molecular weight of starch leads to increasing SAR. The bottom fraction “a” heats better (up to 300 times) than the upper fraction “c.” This improvement is likely due to the larger size of aggregates in the bottom fraction.

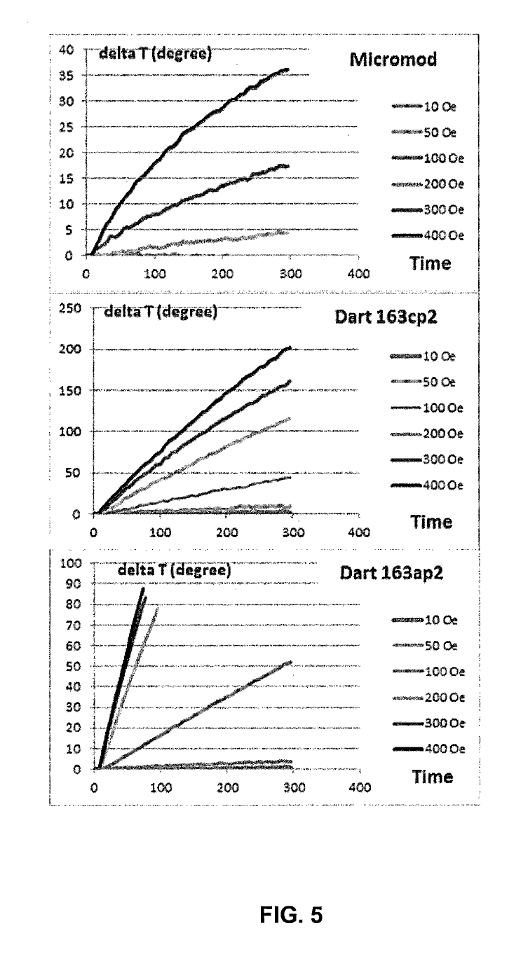

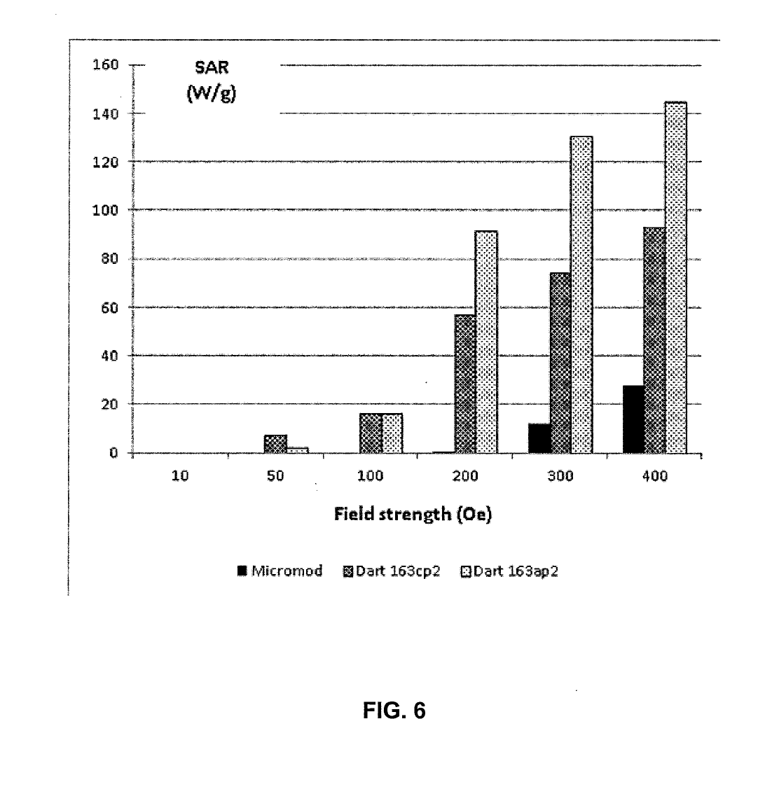

The bottom fraction heats very well (FIG. 5), however it has relatively large aggregates that sediment with time. The upper fraction, which is colloidally stable, produces a moderate amount of heat. The upper fraction of MNP with CM-dex produces significantly more heat, e.g., up to 3 times more heat at 400 Oe, 7 times more at 300 Oe, and 900 times more at 200 Oe) than commercially available analogue and also produces decent amount of heat needed to perform hyperthermia at fields below 200 Oe while commercial available analogues do not produce any heat at that field strength range (FIG. 6).

### EXAMPLE 3

Particle Comparisons

The closest prototype with respect to the presently disclosed instrumentalities is described in Ravikumar et al. [9]. The difference from the presented work includes, but not limited to: chemical composition of precipitate (leads to different physico-chemical properties of the final material), iron salts ratio (leads to different chemical composition of the obtained material), types of organic molecules used (different physico-chemical and biological properties), foreign metal infusion (elaborates uses of obtained material, improves property, makes analyses easier); different order of reactants mixing, temperature treatment and pH lead to different chemical and structural composition of the obtained material such as heating and magnetic properties, size of single crystals and aggregates; different post-synthesis treatment (washing, endotoxin purification and sterilization) make invented particles uniformly size distributed and ready to use in biomedical applications additionally to technical applications. General purpose of prototype materials is fundamental studies (Monte Carlo simulation). General purpose of invented material is applied science (Hyperthermia and other bio-medical application, Material science, Nanotechnology).

Table 1 and FIG. 6 provide a favorable comparison of the SAR values for two sets of nanoparticles that were synthesized according to the instrumentalities disclosed herein, namely, those identified as Dart 163ap2 and Dart 163cp2, versus other nanoparticles obtained on commercial order from “BNF-starch” purchased from Micromode Partikeltechnologie GmbH [10]. The “Dart” nanoparticles show far superior SAR values at any field strength and show SAR values at lower field strengths comparable to values obtained from the commercial nanoparticles at higher field strengths. FIG. 6 shows these results as a graphical comparison.

TABLE 1
OBSERVED SAR(W/G) AT DIFFERENT FIELD STRENGTHS

| Nanoparticle | 10 Oe | 50 Oe | 100 Oe | 200 Oe | 300 Oe | 400 Oe |
|---|---|---|---|---|---|---|
| Micromode | 0 | 0 | 0 | 0.7 | 12 | 28 |
| (BNF-starch) | | | | | | |
| Dart 163ap2 | 0 | 1.7 | 15.9 | 91.2 | 130.6 | 144.5 |
| Dart 163cp2 | 0.5 | 7.3 | 16.1 | 56.7 | 74.2 | 92.7 |

### REFERENCES

The following references are cited above and hereby incorporated by reference to the same extent as though fully replicated herein.

[1] Philosophical Transactions of the Royal Society A: Physical, Mathematical and Engineering Sciences 240 (826): 599-642.

[2] Low viscosity magnetic fluid obtained by the colloidal suspension of magnetite particles: U.S. Pat. No. 3,215,572 USA, H01F/1/10/Stephen, Papell, Solomon. US 19630315096;

[3] Ferrofluid Composition and Process of Mahing Same: U.S. Pat. No. 3,917,538 USA, H01F1/44; H01F1/44; (IPC1-7): H01F1/28; C09D11/00; C10M3/00; H01F1/00/R. E. Rosenazweig; applicant Ferrofluidics Corp. US19730324414 17.

[4] H. Bonnemann, R. A. Brand, W. Brijoux, H.-W. Hofstadt, M. Frerichs, V. Kempter, W. Maus-Friedrichs, N. Matoussevitch, K. S. Nagabhushana, F. Voigts and V. Caps/Air stable Fe and Fe—Co magnetic fluids-synthesis and characterization//Appl. Organometal. Chem. 2005; 19: 790-796

[5] G. Zhang, Y. Liao and I. Baker, Materials Science and Engineering C 30 (1) (2010) 92-97.

[6] K. Buyukhatipoglu, A. Morss Clyne/Controlled flame synthesis of αFe2O3 and Fe304 nanoparticles: effect of flame configuration, flame temperature, and additive loading//Journal of Nanoparticle Research 04/2012; 12(4):1495-1508.

[7] Patent WO 2005/013897 A2//Inventors: Robert Ivkov, Cordula Gruettner, Joachim Teller, Fritz Westphal/Applicant: Triton Biosystems Inc.

[8] J. Phys. D: Appl. Phys. 40 (2007) 320-325

[9] Ravikumar et al., Colloids and Surfaces A: Physicochem. Eng. Aspects 403 (2012) 1-6.

[10] David E. Bordelon, Christine Cornejo, Cordula Gruttner, Fritz Westphal, Theodore L. DeWeese, and Robert Ivkov/Magnetic nanoparticle heating efficiency reveals magneto-structural differences when characterized with wide ranging and high amplitude alternating magnetic fields/JOURNAL OF APPLIED PHYSICS 109,124904 (2011).

1. A method of synthesizing magnetic nanoparticles (MNPs), comprising
forming a solution including iron salts and organic chains, wherein the iron salts include a mixture of Fe(II) and Fe(III);
precipitating, from the iron salts, iron ions in the solution to form iron oxides and iron hydroxides;
hydrothermally developing MNPs in the solution, where each of the MNPs includes a portion of the iron oxides, a portion of the iron hydroxides, and one or more of the organic chains.

2. The method of claim 1, the steps of precipitating and hydrothermally developing cooperating to form each of the MNPs with a crystal matrix structure and one or more of the organic chains interwoven in the crystal matrix structure.

3. The method of claim 1, the step of forming a solution comprising forming the solution with Fe(II):Fe(III) molar ratio greater than 1:2.

4. A nanocomposite comprising one or more of magnetic nanoparticles (MNPs), each of the MNPs including:
iron oxides and iron hydroxides forming a crystal matrix structure; and
organic chains interwoven in the crystal matrix structure.

5. The nanocomposite of claim 4 including ferrites with a dopant moiety selected from the group consisting of Eu, Co, Zn, Mn, Pt and combinations thereof.

6. The nanocomposite of claim 5, wherein the MNPs have an average particle diameter of from 2-5 nm.

7. The nanocomposite of claim 6, wherein the MNPs form aggregates with an average diameter of from 10-100 nm.

8. The nanocomposite of claim 4, wherein the organic chain are selected from the group consisting of carbohydrates, alcohols and glycols having carbon number of at least two, organosilanes, and organosiloxanes.

9. The nanocomposite of claim 4, decorated with a bioactive agent.

10. The nanocomposite of claim 9, wherein the bioactive agent is selected from the group consisting of antibodies, drugs, toxins, markers, and combinations thereof.

11. The nanocomposite of claim 4, the MNPs being dispersed as a colloid in liquid.

12. The nanocomposite of claim 11, wherein the liquid is selected from the group consisting of water, saline solution, plasma, serum, and combinations thereof.

13. The nanocomposite of claim 11, wherein the colloid in liquid is shelf-stable for a period of time exceeding one year.

14. The method of claim 2, wherein the step of hydrothermally developing comprises controlling growth of the MNPs to produce a Z-size of the MNPs predominantly in the range from 70 to 150 nm.

15. The nanocomposite of claim 4, wherein the ratio of Fe(II) to Fe(III) is controlled to produce specific absorption rate (SAR) of the MNPs up to 600.0 W/g in a frequency range from 100 Hz-200 kHz at applied field strengths ranging from 10-1500 Oe.

16. The method of claim 1, further comprising controlling the ratio of Fe(II) to Fe(III) to produce specific absorption rate up to 600.0 W/g in a frequency range from 100 Hz-200 kHz at applied field strengths ranging from 10-1500 Oe.

17. A magnetic nanoparticle (MNP), comprising:
oxides and hydroxides of iron in a ratio of Fe(II):Fe(III) greater than 2:1, such that a plurality of copies of the MNP, suspended as colloids in solution, form aggregates that are responsive in a magnetic field and show specific absorption rate (SARI up to 600.0 W/g in a frequency range from 100 Hz-200 kHz at applied field strengths ranging from 10-1500 Oe.

18. The MNPs of claim 17, wherein the MNP presents a crystal structure that is interwoven with organic chain material to form a nanocomposite.

19. The MNP of claim 18, wherein the nanocomposite is decorated with a bioactive agent.

20. The MNP of claim 19, wherein the bioactive agent is selected from the group consisting of antibodies, drugs, toxins, markers, and combinations thereof.
